(19) United States
(12) Patent Application Publication
(10) Pub. No.: US 2005/0260283 A1
(43) Pub. Date: Nov. 24, 2005

(54) Composition for prevention and treatment of colon adenomas

(75) Inventor: Pardeep Nijhawan, Richmond Hill (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Medical Futures Inc.

(21) Appl. No.: 10/902,069

(22) Filed: Jul. 30, 2004

(30) Foreign Application Priority Data
May 20, 2004 (CA) 2,467,894

(51) Int. Cl. A61K 31/715; A61K 31/525; A61K 31/60; A61K 33/04

(52) U.S. Cl. 424/682; 514/165; 514/54; 514/251; 424/702; 424/643; 514/168; 514/458; 514/474; 514/350; 514/356; 514/275; 514/52; 514/725; 514/763

(57) ABSTRACT

The present invention provides a composition capable of reducing the risk of colon adenoma formation. This composition will be useful for the prevention and treatment of colon cancer through the combination of the ingredients disclosed.

### FIELD OF THE INVENTION

The present invention provides a composition capable of reducing the risk of colon adenoma formation. This composition will be useful for the prevention and treatment of colon cancer through the combination of the ingredients disclosed.

### BACKGROUND OF THE INVENTION

Colon adenomas are precursors to colon cancer, an increasing disease in North America. It is the third most common cancer and cause of mortality in the United States and in other developed countries (Krishnan, 1996). In England and Wales, approximately 29,000 people are diagnosed yearly with 15,000 of those cases being fatal (Courtney, 2004). The colon adenomas that precede cancer are lesions that may be safely removed during colonoscopy; however, if left to their natural course these can lead to colon cancer within 5 to 7 years. Because colon and rectal cancer share many common features and occur closely in the gastrointestinal tract, they are often collectively referred to as colorectal cancer. In attempting to prevent colorectal cancer, attention has been placed on screening and chemoprevention as methods of reducing cases and fatalities from this disease.

Cancer prevention has been defined as the identification of preventable causes of cancer and the reduction of cancer incidence by preventative strategies applied to target populations (Krishnan, 1996). Such forms of cancer prevention have been identified and are sub-classed as primary, secondary or tertiary prevention.

Primary prevention involves identification and elimination of cancer-causing agents such as chemicals, viruses and radiation. Although all of these can contribute to cancer, isolation and attribution to a single factor is difficult. Secondary prevention involves the screening of at-risk individuals, and detection of cancerous growth at early stages using methods such as genetic screening. Tertiary prevention refers to the use of specific pharmacological agents to combat growth and prevent spreading of cancer, and is also know as chemoprevention (Krishnan, 1996).

The pharmaceutical industry is actively involved in tertiary prevention, relying on laboratory and scientific data for the identification and development of potential anticarcinogenic agents.

With primary prevention, exposure-based prevention strategies are restricted by the limited cause-and-effect data available (as with, for example, tobacco and lung cancer). However dietary modification has been regarded as a practical approach to primary prevention, as several mutagens are dietary in origin. Diets high in animal fats and red meats, for example, have been associated with increases in colonic adenomas and colorectal cancer (Giovannucci, 1992; Willet, 1990). The low occurrence of colorectal cancer in Asian countries has been attributed to their high-fibre diets. Because of this, patients of colorectal cancer are often recommended diets of low fat, high fibre, with lots of fruits and vegetables.

Unfortunately, a diet of opposite patterns is becoming more common here in North America. In several studies relating dietary patterns to colorectal cancer risk, the Western diet has been characterized by high intakes of processed and red meats, fast food meats, potatoes, soda and refined grains (Fung, 2003). There are factors which promote such eating habits. The Atkins diet, for example, which suggests a daily intake high in fats and proteins, is becoming increasingly common among dieters. Although the Atkins approach has been useful in helping to reduce some of the obesity issues common in our society, it is also the ideal diet to increase chances of developing colorectal cancer. As noted already, a high fat diet is known to increase the incidence of colon adenoma disease, especially when coming from animal sources such as red meats. It is firmly believed that such a diet, if followed for long periods of time, will increased the incidence of colorectal cancer.

In light of the prevalence and severity of colorectal cancer, it is no surprise that considerable effort has been dedicated towards research, prevention and treatment. Although anyone can get colorectal cancer, at-risk groups as identified above are becoming increasingly common. Hence there is an established need to assist those particularly susceptible to colorectal cancers.

### DESCRIPTION OF THE PRIOR ART

Colorectal cancer has been addressed at great length in both the scientific and patent literature. Various relationships have been examined, and many hypotheses put forward for prevention and treatment. Among the different nutrients and agents suggested to decrease the risk of this disease are anti-inflammatories, antioxidants, calcium, magnesium, polyphenols, vegetables, fruits, fibre and certain vitamins.

The particular components of the composition of the present invention which effect the growth of colon adenomas are aspirin, folic acid, calcium and fibre. Individually, these ingredients have all been considered in their role for prevention of colorectal cancer; however no attempt has been made to combine these proven ingredients in an effective and convenient form for the benefit of potential colorectal patients.

Aspirin has been considered inversely associated with a risk for colorectal polyps for more than a decade. In 2004, Chan et al summarized their findings on 22,077 women aged 34 to 77 by concluding that regular, short-term use of aspirin is inversely associated with a risk for colorectal adenoma formation. Other studies, including those by Chan et al (2003), La Vecchia et al (1997), and Krishnan et al (1997) have made similar conclusions. Unfortunately, aspirin has been causally linked with occurrence of gastrointestinal bleeding making higher dosages of this drug undesirable (Chan, 2004). Although it has been established from epidemiological studies that a 40-50% reduction in the incidence of colorectal cancer can be due to regular aspirin use, the optimal dose and frequency of administration cannot be deduced (Krishnan, 1997).

U.S. Pat. No. 6,231,888 B1 claims the use of an NSAID or cox-2 or cox-1-specific inhibitor in a delivery system specific to the colon. This invention is taught to directly inhibit colon polyps, and includes within its scope aspirin and several other NSAIDs. The patent invention in addition to aspirin includes within its scope other agents for the treatment and prevention of colon polyps, however it does not include the components of the present invention.

Folic acid, also known as folate, has been considered alone in the scientific literature for the treatment of colorectal cancer. While the mechanism of action of this agent is not well understood, the long-term use of this nutrient as a dietary supplement has been shown to reduce the risk of colorectal adenomas. Intake of folate, or fruits and vegetables, the major sources of folate in most populations has been associated with a reduction in risk of colorectal neoplasia in several studies (Giovannucci, 1993).

While not wishing to be bound by theory, applicant notes recent research where deficiency in folate was taught to disrupt DNA methylation and eventually DNA synthesis, leading to colorectal neoplasms (Nagothu, 2003). Colorectal neoplasms can lead to colon adenomas. Nagothu and coworkers noted that restoration of DNA methylation status occurred simultaneously with supplementation of folic acid. They further noted that folic acid affects other intracellular events that critically relate to cell growth, lending further support to the current invention. Regardless of the mechanism of action, folic acid supplementation has been shown to reduce risk of colon adenoma formation and is incorporated herein for such use.

Calcium has been considered alone and in combination with other agents for the prevention of colorectal adenomas. Baron and coworkers considered this hypothesis by administering calcium carbonate to volunteers with a history of colorectal adenomas. It was concluded that calcium supplementation was associated with a moderate but significant reduction in the risk of recurrent colorectal adenomas. In summary, however, these researchers acknowledged the uncertainty of calcium intake when combined with other influential factors such as dietary fat, vitamins and mineral supplements or aspirin (Baron, 1999). The present invention has addressed that need in an innovative product.

Present scientific research has suggested that calcium acts by binding potential carcinogens in the lumen of the gut or, alternatively, by directly controlling epithelial function. Calcium is known to have a high affinity for complexing bile salts, and this has also been suggested to prevent salts from interacting with colonic epithelium, reducing events that lead to colon adenoma. In yet another hypothesis, calcium regulates critical cell-cell interactions, affected by calcium receptors. A review of these mechanisms has been published by Umar et al (2003). In any regard calcium supplementation has been confidently correlated with a reduction in the risk of colorectal cancer.

U.S. Pat. No. 6,251,439 B1 is for the administration of elemental calcium for reducing the risk of carcinogenesis and is directed towards colorectal adenoma formation. While it is claimed for daily administration in the carbonate, citrate, hydroxide, phosphate or chlorophosphate salt forms, its combination with any other proven agents for the benefit of potential colorectal cancer patients is not taught.

Calcium is further included as it is known to decrease the incidence of gastrointestinal bleeding common to habitual use of aspirin. It is anticipated that the combination of aspirin with elemental calcium along with certain vitamins will provide a gastro-protective effect, countering the toxicity of aspirin.

Fibre has been considered as a nutrient beneficial for the prevention of colorectal cancer. Both the American Cancer Society and the National Cancer Institute recommend consumption of at least 5 servings of fruit and vegetables and 20-30 grams of dietary fibre per day (Kim, 2000). Although a number of studies have shown this correlation, the review of Krishnan provides enough support by summarizing studies wherein wheat fibre supplements were shown to reduce the number and sizes of rectal adenomas (Krishnan, 1996). Fibre is thought to provide a beneficial effect by increasing the stool bulk, binding potential carcinogens, lowering faecal pH and promoting a favourable colonic microflora (Kim, 2000). Regardless of mechanism this agent has been associated with reduced risk of colon adenoma formation and is incorporated herein for such use. The preferred form of fibre to be used with this invention is soluble fibre from fruits or vegetables. The invention therefore may take the form of pills, tablets, capsules, sachets or the like. Other forms of fibre may also be appropriate.

Patient compliance is an issue that has a great effect on the outcome of any treatment. For nearly a half decade compliance has been researched for its effects on drug therapy. Evidence has been consistent in showing that drug regimens involving multiple components reduce compliance. In particular, adherence to drug regimens drops off sharply when patients are administered 3 or more drugs per day (Blackwell, 1979).

With primary prevention of colorectal cancer, many agents have been shown to display only moderate effects against development. It would therefore be prudent for at-risk groups to consider the habitual consumption of more than one of these agents for the reduction in risk of colorectal cancer.

In light of the issues related to compliance, it would be beneficial to provide combination compositions as a method of treatment. It is in this regard that the composition of the present invention will find its greatest benefit for potential colorectal cancer patients.

Combinations of agents for the prevention of colorectal cancer have previously been disclosed in the art. U.S. Pat. No. 6,703,380 B2 is for the combination of a cox inhibitor, calcium and vitamin D3 for the prevention of cancer, particularly colorectal cancer. Similarly, U.S. Pat. No. 6,646,013B1 is for a combination of calcium carbonate and folic acid useful in the prevention and reduction of colon rectal cancer. These combinations, while perhaps useful for treatment of colorectal adenomas, lack the efficacy and convenience of the present invention. The combined administration of four proven agents for the treatment and prevention of colorectal cancer is unique as it will promote efficacy through compliance among patients.

Among scientific literature, Courtney et al (2003) review the state of the art in the field of colorectal cancer. Many agents are reviewed here individually as effective for the treatment of colorectal cancer, including aspirin, folic acid, calcium and fibre which, in combination, are ingredients of the present invention. Without suggesting effective dosages regimens for these or any other ingredients, the research summarized in this article establishes a long felt need for a single agent capable of reducing the high mortality due to colorectal cancer.

### REFERENCES

Barker, A D, et al, U.S. Pat. No. 6,646,013 B1
Baron, J A, et al, N Engl J Med, (1999); 340(2): 101-7
Blackwell, B, The Drug Regimen and Treatment Compliance, Chapter 9 in “Compliance in Healthcare”, eds R B Haynes, D W Taylor and D L Sackett, Johns Hopkins University Press, Baltimore, Md., 1979.
Chan, A T, et al, Ann Intern Med 2004; 140:157-66
Chan A T, Cancer Causes and Control, (2003) 14:413-8
Courtney, E D J, et al, Aliment Pharmacol Ther, (2004); 19:1-24
Fung, T, et al. Arch Int Med, 2003;163:309-314
Giovannucci, E, et al, J Natl Cancer Inst (1992) 84: 91-8.
Giovannucci, E, et al, J Natl Cancer Inst (1993) 85: 875-884
Giovannucci, E, et al, Ann Intern Med, (1994) 121:241-246
Heibel, R, et al, U.S. Pat. No. 6,274,170 B1
Holt, S, et al, J Allergy Clin Immunol, (2004), 113:219-220
Kim, Y I, Gastroenterology 2000; 118: 1235-57
Krishnan, K, et al, J Cell Biochem Suppl 28/29:148-158 (1997)
Krishnan, K. Gastroenterol Clin North Am, (1996) 25(4); 821-58
La Vecchia, C, et al. Br J Cancer, (1997), 76(5); 675-7
Lerner, E I, et al, U.S. Pat. No. 6,231,888B1
Melikian, C, et al, Clin Ther, (2002) 24:460-467
Nagothu, K K, Cancer Detection and Prevention, (2003) 27:297-304
Negri, E, et al, Nutr Cancer, (1990); 13(4): 255-62
O'Keefe, S J D, Am J Gastroent, (1999), 94:1373-1380
Pritchard M D, et al, Pharmacol Ther, (1996), 72:149-169
Raskov, U.S. Pat. No. 6,703,380 B2
Riley, P A, et al, U.S. Pat. No. 5,948,443, et al
Slattery, M L, et al, Am J Clin Nutr, (2004) 79:274-81
Steinmetz, K A, et al, J Am Diet Assoc, (1996), 1027-1039
Trock, B, et al, J Natl Cancer Inst, (1990), 82:650
Umar, S, et al, Cell Prolif, (2003), 36:361-75
Vester, S R, U.S. Pat. No. 6,203,818 B1
Weissman, D L, et al, U.S. Pat. No. 6,121,249
Weissman, D L, U.S. Pat. No. 6,323,188 B1
Willet W C, et al, N Engl J Med 1990; 323: 1664-72.

### SUMMARY OF THE INVENTION

In light of the increasing rate of colorectal cancer, there is an established need for a composition useful for prevention of colorectal adenomas. While individual agents have been evaluated for their efficacy against this disease, there has been no attempt to combine several of these agents together for the benefit of patients. Such a composition would enable the delivery of several active agents in a single administration, encouraging compliance and discouraging colon adenoma growth.

With poor diet habits becoming more common, there are individuals particularly susceptible to colorectal cancer, expressing an even greater need for protection. It is therefore an objective of the present invention to provide a composition designed to reduce the occurrence and growth of colon adenomas for these and other patients. This composition is designed to optimize effective agents known to combat colon adenomas in order to address an evident and growing need.

The ingredients included as preventative agents within the present invention include in combination a therapeutically effective amount of aspirin, folic acid, calcium and fibre. This combination is designed such that the ingredients compliment the actions of one another while providing patients with a convenient and effective method of delivery. The provision of these ingredients in one package will increase patient compliance, effectively decreasing risk of adenoma development.

Therefore, in one aspect the present invention is useful in the prevention of colorectal cancer through the therapeutically effective combination of calcium, folic acid, fibre and aspirin for the prevention of formation of colorectal adenomas.

Preferably, the present invention is also formulated as lactose-free, allowing for easy absorption without causing bloating or any other malabsorption symptoms. Additionally, compositions of the current invention are gluten-free, also designed to prevent malabsorption.

In another aspect of the present invention, the current formulation is to be administered orally, taking the form of a pill, tablet, capsule, caplet, or sachet. A kit comprising a powder sachet version is preferred and may be formulated for administration to those whose needs entail a high-fibre version. Alternatively the composition may be ingested in pill, tablet, capsule or caplet form in multiple numbers at a time to provide the required fibre dosage. Soluble fibres, such as fruit and vegetable fibres are preferred.

In yet another aspect of the embodiments of the invention described in the above paragraphs, additional components may be included for their nutritional and dietary value, having neither a beneficial nor known detrimental effect for prevention of colorectal adenomas.

These can include:
Vitamin A
Beta Carotene
Vitamin D3
Vitamin B1
Vitamin B2
Vitamin B6
Vitamin B12
Niacinamide
Vitamin C
Pantothenic Acid
Iron
Zinc
Vitamin E
Selenium
Iodine
Vitamin K
Copper
Chromium
Magnesium

According to one aspect of the invention, there is provided a composition comprising a therapeutically effective amount of aspirin, folic acid, calcium and fibre at amounts sufficient for the prevention and treatment of colon adenomas.

In another aspect of the present invention, there is provided a composition wherein aspirin is present in amounts of between 25 mg to 325 mg.

In another aspect of the present invention, there is provided a composition wherein folic acid is present in amounts of between 0.1 mg to 5.0 mg

In another aspect of the present invention, there is provided a composition wherein calcium is present in amounts of between 100 mg to 1000 mg.

In another aspect of the present invention, there is provided a composition wherein fibre is present in amounts of between 0.1 to 10 g.

In another aspect of the present invention, there is provided a composition wherein additional vitamins, nutrients or non-medicinal components are formulated for effects not related to risk reduction in the formation of colon adenomas. Such ingredients may include, but are not limited to, Vitamin A, beta carotene, vitamin D3, vitamin B1, vitamin B2, vitamin B6, vitamin B12, niacinamide, vitamin c, pantothenic acid, iron, zinc, vitamin E, selenium, iodine.

In another aspect of the present invention, there is provided a composition of the above description, or any pharmaceutically acceptable salt thereof; additionally said composition may be formulated with any appropriate delivery vehicle.

In another aspect of the present invention, there is provided a composition administered in the form of a capsule, tablet, caplet or powder formulation or in a liquid dosage form.

In another aspect of the present invention, there is provided a composition intended as a method of treating and preventing formation of colon adenomas in high-risk patients, including those on high fat, high protein and low fibre diets, comprising the administration of a composition of any of the prior descriptions.

In another aspect of the present invention, there is provided a method, as previously described wherein the composition is administered once, twice or three times daily.

### EXAMPLES

The following examples are given to illustrate the invention, without limiting the scope thereof.

### Example 1

A preferred example provides a tablet for colon adenoma reduction. The present example is the low fibre dosage. The preferred administration is two to three tablets daily.
Calcium Carbonate 600 mg
Folic Acid 0.5 mg
Aspirin 80 mg
Fibre 0.5 g

Non-medicinal ingredients include, but are not limited to:
Vitamin A
Beta Carotene
Vitamin D3
Vitamin B1
Vitamin B2
Vitamin B6
Vitamin B12
Niacinamide
Vitamin C
Pantothenic Acid
Iron
Zinc
Vitamin E
Selenium
Iodine

### Example 2

A preferred example provides a tablet for colon adenoma reduction with the high fibre dosage version. The preferred administration is two to three tablets daily along with a sachet of fibre for reconstitution in 240 ml of water or juice. Alternatively, the calcium, folic acid, aspirin and fibre may be contained in a sachet to be consumed three times per day.
Calcium Carbonate 600 mg
Folic Acid 0.5 mg
Aspirin 80 mg

Non-medicinal ingredients include, but are not limited to:
Vitamin A
Beta Carotene
Vitamin D3
Vitamin B1
Vitamin B2
Vitamin B6
Vitamin B12
Niacinamide
Vitamin C
Pantothenic Acid
Iron
Zinc
Vitamin E
Selenium
Iodine
Sachet of 10 g Fibre in a soluble pleasant citrus flavour.

### Example 3

A preferred example provides a sachet for reconstitution in 240 ml of water or juice with the high fibre dose version. The sachet would contain the following:
Calcium Carbonate 1200 mg
Folic Acid 0.5 mg
Aspirin 300 mg
Fibre 10 gm in a soluble pleasant citrus flavour

Non-medicinal ingredients include, but are not limited to:
Vitamin A
Beta Carotene
Vitamin D3
Vitamin B1
Vitamin B2
Vitamin B6
Vitamin B12
Niacinamide
Vitamin C
Pantothenic Acid
Iron
Zinc
Vitamin E
Selenium
Iodine

As may, changes can be made to the embodiments of the invention without departing from the scope thereof. It is intended that all matters contained herein be considered illustrative of the invention and not in a limiting sense.

1. A composition comprising a therapeutically effective amount of aspirin, folic acid, calcium and fibre at amounts sufficient for the prevention and treatment of colon adenomas.

2. The composition, according to claim 1, wherein aspirin is present in amounts of between 25 mg to 325 mg.

3. The composition, according to claim 1, wherein folic acid is present in amounts of between 0.1 mg to 5.0 mg

4. The composition according to claim 1, wherein calcium is present in amounts of between 100 mg to 1000 mg.

5. The composition, according to claim 1, wherein fibre is present in amounts of between 0.1 to 10 g.

6. The composition according to claim 1 including additional vitamins, nutrients or non-medicinal components, formulated for effects not related to risk reduction in the formation of colon adenomas; such ingredients may include, but are not limited to, Vitamin A, beta carotene, vitamin D3, vitamin B1, vitamin B2, vitamin B6, vitamin B12, niacinamide, vitamin c, pantothenic acid, iron, zinc, vitamin E, selenium, iodine.

7. The composition according to claim 6, or any pharmaceutically acceptable salt thereof.

8. The composition according to claim 7, formulated with any appropriate delivery vehicle.

9. The composition according to claim 8, useful in the reduction in risk of colon adenoma formation.

10. The composition according to claim 9, wherein said composition is administered in the form of a capsule, tablet, caplet or powder formulation.

11. The composition according to claim 9, wherein said composition is administered in liquid dosage form.

12. A method of treating and preventing formation of colon adenomas in high-risk patients, including those on high fat, high protein and low fibre diets, comprising the administration of a composition of any of the previous claims

13. A method, according to claim 12 wherein the composition is administered once, twice or three times daily.
